(19) United States
(12) Patent Application Publication
(10) Pub. No.: US 2023/0085607 A1
(43) Pub. Date: Mar. 16, 2023

(54) IVERMECTIN COMPOSITIONS FOR TREATMENT OF COVID-19

(71) Applicant: Therapeutic Solutions International, Inc., Oceanside, CA (US)

(72) Inventors: Thomas E. Ichim, Oceanside, CA (US); Famela Ramos, Oceanside, CA (US); James Veltmeyer, Oceanside, CA (US); Timothy G. Dixon, Oceanside, CA (US); Feng Lin, Oceanside, CA (US); Wais Kaihani, Oceanside, CA (US)

(73) Assignee: Therapeutic Solutions International, Inc., Oceanside, CA (US)

(21) Appl. No.: 17/931,872

(22) Filed: Sep. 13, 2022

Related U.S. Application Data
(60) Provisional application No. 63/245,051, filed on Sep. 16, 2021.

(51) Int. Cl. A61K 31/7048; A61K 31/353; A61K 31/09; A61K 31/122; A61K 31/26; A61K 36/82; A61K 36/45; A61K 36/71; A61K 36/31

(52) U.S. Cl. CPC A61K 31/7048; A61K 31/353; A61K 31/09; A61K 31/122; A61K 31/26; A61K 36/82; A61K 36/45; A61K 36/71; A61K 36/31

(57) ABSTRACT

Disclosed are novel mechanisms of action of ivermectin therapy as related to treatment of COVID-19 and means of augmenting therapeutic activities by co-administration with one or more of the following: pterostilbene, thymoquinone, epigallocatechin-3-gallate, and sulforaphane. In one embodiment the invention provides enhanced reduction of inflammation induced pulmonary leakage without augmenting immune suppressive mechanisms.

### CROSS-REFERENCE TO RELATED APPLICATIONS

This application is a non-provisional of and claims priority to U.S. Provisional Application Ser. No. 63/245,051, filed Sep. 16, 2021, which is hereby incorporated by reference in its entirety.

### FIELD OF THE INVENTION

The invention pertains to the field of immune modulatory/antiviral therapeutics, more specifically, the invention pertains to therapeutic compositions capable of enhancing effects of ivermectin in reducing COVID-19 pathology.

### BACKGROUND

Severe acute respiratory syndrome coronavirus-2 (SARS-CoV-2) is a novel viral strain belonging to the coronavirus family. The main strains of this family are 229E (alpha coronavirus) [1-6], NL63 (alpha coronavirus) [7-21], OC43 (beta coronavirus) [22-24], and HKU1 (beta coronavirus) [25-30], which are relatively innocuous and cause the common cold, as well as more virulent strains such as MERS-CoV (the beta coronavirus that causes Middle East Respiratory Syndrome, or MERS) [31-36], SARS-CoV (the beta coronavirus that causes severe acute respiratory syndrome, or SARS) [37], and SARS-CoV-2 (the novel coronavirus that causes coronavirus disease 2019, or COVID-19) [38].

Most of the cases are acute with symptoms like fever, shortness of breath and fatigue. However, growing evidence suggests that higher mortality is associated with further longer-term health complications. Clinical manifestations of SARS-CoV-2 have been reported in which patients develop flu or pneumonia-like respiratory syndrome along with organ damage such as liver and heart A recent work has shown that ACE2 (host cell receptor for SARS-CoV-2 entry) expression levels do not differ based on age, sex or ethnicity 5. This part explains the wide-spread transmission of the virus and raises the possibility of immune response as critical factor for mortality risk. Indeed, high level of pro-inflammatory cytokines were evident both in Middle East Respiratory Syndrome (MERS) coronavirus and Severe Acute Respiratory Syndrome coronavirus (SARS-CoV) infection. This results in the infiltration of immune cells, thereby promoting lung injury. Severely ill COVID-19 patients also demonstrated higher levels of pro-inflammatory cytokines in bronchoalveolar lavage fluid (B ALF) and peripheral blood mononuclear cells (PBMC). In addition, most of the COVID-19 patients exhibited an increase in inflammatory monocytes and neutrophils.

### SUMMARY

Preferred embodiments include methods of augmenting the prophylactic and/or therapeutic effects of ivermectin on COVID-19 comprising administering said ivermectin together with one or more natural ingredients selected from a group comprising of: a) Green Tea and/or extract thereof; b) Blueberry and/or extract thereof; c) Nigella sativa and/or extract thereof; and d) broccoli and/or extract thereof.

Preferred methods include embodiments wherein said green tea extract is epigallocatechin-3-gallate or an analogue thereof.

Preferred methods include embodiments wherein said blueberry extract is pterostilbene or an analogue thereof.

Preferred methods include embodiments wherein said Nigella sativa extract is thymoquinone or an analogue thereof.

Preferred methods include embodiments wherein said broccoli extract is sulforaphane or an analogue thereof.

Preferred methods include embodiments wherein said therapeutic combination is administered at a dosage and frequency sufficient to inhibit lung protective effects of ivermectin.

Preferred methods include embodiments wherein said lung protective effects are reduction of inflammatory cytokines in the lung.

Preferred methods include embodiments wherein said inflammatory cytokines are selected from a group comprising of: a) interleukin-1; b) interleukin-2; c) interleukin-5; d) interleukin-6; e) interleukin-8; f) interleukin-9; g) interleukin-11; h) interleukin-12; i) interleukin-15; j) interleukin-16; k) interleukin-17;1) interleukin-18; m) interleukin-21; n) interleukin-22; o) interleukin-23; p) interleukin-25; q) interleukin-27; r) interleukin-33; s) TNF-alpha; t) TNF-beta; u) interferon alpha; v) interferon beta; w) interferon gamma; x) interferon tau; y) interferon omega.

Preferred methods include embodiments wherein said lung protective effect is reduction of neutrophil infiltration into the lung.

Preferred methods include embodiments wherein said lung protective effect is reduction of T cell infiltration into the lung.

Preferred methods include embodiments wherein said T cell is a gamma delta T cell.

Preferred methods include embodiments wherein said T cell is a CD4 T cell.

Preferred methods include embodiments wherein said CD4 T cell is a Th1 cell.

Preferred methods include embodiments wherein said Th1 cell expresses STAT1.

Preferred methods include embodiments wherein said Th1 cell expresses STAT4.

Preferred methods include embodiments wherein said Th1 cell expresses T-bet.

Preferred methods include embodiments wherein said Th1 cell expresses CCR1.

Preferred methods include embodiments wherein said Th1 cell expresses CCR.

Preferred methods include embodiments wherein said Th1 cell expresses CXCR3.

Preferred methods include embodiments wherein said Th1 cell expresses CD119.

Preferred methods include embodiments wherein said Th1 cell expresses interferon gamma receptor 2.

Preferred methods include embodiments wherein said Th1 cell expresses interleukin-10 receptor.

Preferred methods include embodiments wherein said Th1 cell expresses CD25.

Preferred methods include embodiments wherein said Th1 cell expresses interleukin-12 receptor alpha.

Preferred methods include embodiments wherein said Th1 cell expresses interleukin-12 receptor beta.

Preferred methods include embodiments wherein said Th1 cell expresses interleukin-18 receptor alpha.

Preferred methods include embodiments wherein said Th1 cell expresses interleukin-18 receptor beta.

Preferred methods include embodiments wherein said Th1 cell expresses interleukin-27 receptor alpha.

Preferred methods include embodiments wherein said Th1 cell expresses interleukin-27 receptor beta.

Preferred methods include embodiments wherein said Th1 cell expresses interleukin-33 receptor alpha.

Preferred methods include embodiments wherein said Th1 cell secretes interleukin-1.

Preferred methods include embodiments wherein said Th1 cell secretes interleukin-2.

Preferred methods include embodiments wherein said Th1 cell secretes interleukin-7.

Preferred methods include embodiments wherein said Th1 cell secretes interleukin-8.

Preferred methods include embodiments wherein said Th1 cell secretes interleukin-12.

Preferred methods include embodiments wherein said Th1 cell secretes interleukin-15.

Preferred methods include embodiments wherein said Th1 cell secretes interleukin-16.

Preferred methods include embodiments wherein said Th1 cell secretes interleukin-18.

Preferred methods include embodiments wherein said Th1 cell secretes interleukin-33.

Preferred methods include embodiments wherein said Th1 cell secretes TNF-alpha.

Preferred methods include embodiments wherein said Th1 cell secretes TNF-beta.

Preferred methods include embodiments wherein said Th1 cell secretes interferon gamma.

Preferred methods include embodiments wherein said CD4 T cell is a Th2 cell.

Preferred methods include embodiments wherein said Th2 cell produces interleukin-4.

Preferred methods include embodiments wherein said Th2 cell produces interleukin-2.

Preferred methods include embodiments wherein said Th2 cell produces interleukin-10.

Preferred methods include embodiments wherein said Th2 cell produces interleukin-13.

Preferred methods include embodiments wherein said Th2 cell produces interleukin-14.

Preferred methods include embodiments wherein said Th2 cell produces interleukin-20.

Preferred methods include embodiments wherein said Th2 cell produces interleukin-35.

Preferred methods include embodiments wherein said Th2 cell produces VEGF.

Preferred methods include embodiments wherein said Th2 cell produces EGF.

Preferred methods include embodiments wherein said Th2 cell produces IGF.

Preferred methods include embodiments wherein said Th2 cell produces FGF-1.

Preferred methods include embodiments wherein said Th2 cell produces FGF-2.

Preferred methods include embodiments wherein said Th2 cell produces FGF-5.

Preferred methods include embodiments wherein said Th2 cell promotes immunity towards extracellular bacteria.

Preferred methods include embodiments wherein said Th2 cell promotes immunity towards parasites.

Preferred methods include embodiments wherein said Th2 cell expresses GATA-3.

Preferred methods include embodiments wherein said Th2 cell expresses IRF-4.

Preferred methods include embodiments wherein said Th2 cell expresses STATS5.

Preferred methods include embodiments wherein said Th2 cell expresses STAT6.

Preferred methods include embodiments wherein said Th2 cell expresses CCR8.

Preferred methods include embodiments wherein said Th2 cell expresses IL-4 receptor alpha.

Preferred methods include embodiments wherein said Th2 cell expresses IL-4 receptor beta.

Preferred methods include embodiments wherein said Th2 cell expresses IL-17 receptor alpha.

Preferred methods include embodiments wherein said Th2 cell expresses IL-17 receptor beta.

Preferred methods include embodiments wherein said Th2 cell expresses IL-33 receptor.

Preferred methods include embodiments wherein said Th2 cell expresses TSLP receptor.

Preferred methods include embodiments wherein said Th2 cell expresses IL-13 receptor alpha.

Preferred methods include embodiments wherein said Th2 cell expresses IL-4 receptor beta.

Preferred methods include embodiments wherein said CD4 T cell is a Th17 T cell.

Preferred methods include embodiments wherein said Th17 T cell is capable of stimulating production of TNF-alpha from naïve T cells.

Preferred methods include embodiments wherein said Th17 T cell is capable of stimulating production of TNF-alpha from myeloid lineage cells.

Preferred methods include embodiments wherein said myeloid lineage cells are myeloid derived suppressor cells.

Preferred methods include embodiments wherein said myeloid lineage cells are monocytes.

Preferred methods include embodiments wherein said myeloid lineage cells are Kupffer cells.

Preferred methods include embodiments wherein said myeloid lineage cells are scar associated macrophage cells.

Preferred methods include embodiments wherein said myeloid lineage cells are lipid associated macrophage cells.

Preferred methods include embodiments wherein said myeloid lineage cells are glial cells.

Preferred methods include embodiments wherein said Th17 T cell is capable of stimulating production of TNF-alpha from endothelial cells.

Preferred methods include embodiments wherein said Th17 T cell is capable of stimulating production of TNF-alpha from pulmonary epithelial cells.

Preferred methods include embodiments wherein said Th17 T cells produce interleukin-17A.

Preferred methods include embodiments wherein said Th17 T cells produce interleukin-17F.

Preferred methods include embodiments wherein said Th17 T cells produce CCL20.

Preferred methods include embodiments wherein said Th17 T cells produce interleukin-21

Preferred methods include embodiments wherein said Th17 T cells produce interleukin-22

Preferred methods include embodiments wherein said Th17 T cells produce interleukin-26

Preferred methods include embodiments wherein said Th17 T cells express BatF.

Preferred methods include embodiments wherein said Th17 T cells express IRF4.

Preferred methods include embodiments wherein said Th17 T cells express ROR-alpha.

Preferred methods include embodiments wherein said Th17 T cells express ROR-gamma.

Preferred methods include embodiments wherein said Th17 T cells express STAT3.

Preferred methods include embodiments wherein said Th17 T cells express CCR4.

Preferred methods include embodiments wherein said Th17 T cells express CCR6.

Preferred methods include embodiments wherein said Th17 T cells express IL-1 receptor alpha.

Preferred methods include embodiments wherein said Th17 T cells express IL-1 receptor beta.

Preferred methods include embodiments wherein said Th17 T cells express IL-6 receptor alpha.

Preferred methods include embodiments wherein said Th17 T cells express IL-6 receptor beta.

Preferred methods include embodiments wherein said Th17 T cells express IL-21 receptor.

Preferred methods include embodiments wherein said Th17 T cells express IL-23 receptor.

Preferred methods include embodiments wherein said TGF-beta receptor II.

Preferred embodiments include methods of reducing inflammation associated hypercoagulation states comprising administration of a therapeutic combination comprising of: a) Green Tea and/or extract thereof;

b) Blueberry and/or extract thereof; c) Nigella sativa and/or extract thereof; d) broccoli and/or extract thereof and e) ivermectin.

Preferred methods include embodiments wherein said green tea extract is epigallocatechin-3-gallate or an analogue thereof.

Preferred methods include embodiments wherein said blueberry extract is pterostilbene or an analogue thereof.

Preferred methods include embodiments wherein said Nigella sativa extract is thymoquinone or an analogue thereof.

Preferred methods include embodiments wherein said broccoli extract is sulforaphane or an analogue thereof.

Preferred methods include embodiments wherein said therapeutic combination is administered at a dosage and frequency sufficient to inhibit tissue factor expression.

Preferred methods include embodiments wherein said tissue factor expression is on the endothelium.

Preferred methods include embodiments wherein said tissue factor expression is on microglia.

Preferred methods include embodiments wherein said tissue factor expression is on the monocytes.

Preferred methods include embodiments wherein said tissue factor expression is on pulmonary endothelium.

Preferred methods include embodiments wherein said tissue factor expression is on the renal endothelium.

Preferred methods include embodiments wherein said therapeutic combination is QuadraMune™.

Preferred methods include embodiments wherein said QuadraMune is administered at a concentration of 10 mg to 10 grams per day.

Preferred methods include embodiments wherein said QuadraMune is administered at a concentration of 100 mg to 2 grams per day.

Preferred methods include embodiments wherein said QuadraMune is administered at a concentration of 200 mg to 1 gram per day.

Preferred methods include embodiments wherein said hypercoagulation state is caused by viral infection.

Preferred methods include embodiments wherein said therapeutic mixture decreases hypercoagulability state by inducing upregulated expression of thrombomodulin.

Preferred methods include embodiments wherein said therapeutic mixture decreases hypercoagulability state by inducing upregulated expression of anti-thrombin III.

Preferred methods include embodiments wherein said therapeutic mixture decreases hypercoagulability state by inducing upregulated expression of Protein C

### BRIEF DESCRIPTION OF THE DRAWINGS

FIG. 1 is a bar graph showing capable of enhancing effects of ivermectin in reducing COVID-19 pathology.

### DETAILED DESCRIPTION OF THE INVENTION

COVID-19 lethality has been shown to be associated with dysregulated cytokine production [39]. For example, inflammatory markers such as IL-6 [40-42], IL-17 [43-50], D-Dimer [51, 52], erythrocyte sedimentation rate [53], have been shown to correlate with severity of disease. Systemic immune activation is also see with the increase in the neutrophil extracellular traps (NETS) that is observed in patients with severe disease [54].

Supporting a role for IL-6 in causing COVID-19 pathology are studies showing that antibody blockade of this cytokine has some level of therapeutic benefit [55-61].

It is known that cytokine manipulation can be performed for therapeutic gain using antibodies, blocking molecules or soluble receptors [62].

Indirect means of decreasing IL-6, such as consumption of vitamin D3 have also been reported [63].

Another interesting point is that COVID-19 severity and mortality correlate with increases in T regulatory cells as assessed by expression of FoxP3 [64].

In some embodiments of the invention, ivermectin is administered together with the therapeutic composition comprising of ivermectin together with one of more of the following: pterostilbene, thymoquinone, EGCG, and sulforaphane, or a pharmaceutically acceptable salt thereof.

The therapeutic dosage is based on several parameters associated with the outcome desired. For example, in certain situations enhancement of various aspects of immunity in a patient suffering from COVID-19 is desired. Based on assessment of immunity, dosing and/or frequency of the therapeutic product may be altered. On some embodiments, composition of the therapeutic factors may be altered based on desired outcome. For example, if a potent endothelial protective effect is desired, the practitioner of the invention may increase the amount of pterostilbene in the composition. The effects of pterostilbene on the endothelium are known and incorporated by reference [65-70]. In other embodiments, if increase lung protective properties are desired, increased concentration of sulforaphane may be performed. The utilization of sulforaphane for pulmonary protection has been previously described and is incorporated by reference [71 -74].

The therapeutic compositions of the invention can be prepared by conventional procedures for blending and mixing compounds. Preferably, the composition also includes an excipient, most preferably a pharmaceutical excipient. Compositions containing an excipient and incorporating the pterostilbene can be prepared by procedures known in the art. For example, the ingredients can be formulated into tablets, capsules, powders, suspensions, solutions for oral administration and solutions for parenteral administration including intravenous, intradermal, intramuscular, and subcutaneous administration, and into solutions for application onto patches for transdermal application with common and conventional carriers, binders, diluents, and excipients.

While a chemical compound of the invention for use in therapy may be administered in the form of the raw chemical compound, it is preferred to introduce the active ingredient, optionally in the form of a physiologically acceptable salt, in a pharmaceutical composition together with one or more adjuvants, excipients, carriers, buffers, diluents, and/or other customary pharmaceutical auxiliaries.

In a preferred embodiment, the invention provides pharmaceutical compositions comprising the chemical compound of the invention, or a pharmaceutically acceptable salt or derivative thereof, together with one or more pharmaceutically acceptable carriers, and, optionally, other therapeutic and/or prophylactic ingredients, known and used in the art. The carrier(s) must be “acceptable” in the sense of being compatible with the other ingredients of the formulation and not harmful to the recipient thereof.

The invention is practiced using components such as pterostilbene. Pterostilbene (trans-3,5-dimethoxy-4-hydroxystilbene) is a natural polyphenolic compound, primarily found in fruits, such as blueberries, grapes, and tree wood. It has been demonstrated to possess potent antioxidant and anti-inflammatory properties. It is a dimethylated analog of resveratrol which is found in blueberries [75], and is believed to be one of the active ingredients in ancient Indian Medicine [76]. The pterostilbene molecule is structurally similar to resveratrol, the antioxidant found in red wine that has comparable anti-inflammatory, and anticarcinogenic properties; however, pterostilbene exhibits increased bioavailability due to the presence of two methoxy groups which cause it to exhibit increased lipophilic and oral absorption [77-81]. In animal studies, pterostilbene was shown to have 80% bioavailability compared to 20% for resveratrol making it potentially advantageous as a therapeutic agent [77].

We have demonstrated the pterostilbene administered in the form of NanoStilbene in cancer patients results in increased NK cell activity, as well as interferon gamma production. Additionally, pterostilbene has shown to inhibit inflammatory cytokines associated with ARDS. For example, studies have demonstrated inhibition of interleukin-1 [82], interleukin-6 [83, 84], interleukin-8 [85], and TNF-alpha [86], by pterostilbene.

First. Taking Kalonji increases the potency of the immune system [87, 88]. Specifically, it has been shown that kalonji activates the natural killer cells of the immune system. Natural killer cells, also called NK cells are the body's first line of protection against viruses. It is well known that patients who have low levels of NK cells are very susceptible to viral infections. Kalonji has been demonstrated to increase NK cell activity. In a study published by Dr. Majdalawieh from the American University of Sharjah, Sharjah, United Arab Emirates [89], it was shown that the aqueous extract of Nigella sativa significantly enhances NK cytotoxic activity. According to the authors, this supports the idea that NK cell activation by Kalonji can protect not only against viruses but may also explain why some people report this herb has activity against cancer. It is known that NK cells kill virus infected cells but also kill cancer cells. There are several publications that show that Kalonji has effects against cancer [90-104].

Second. Kalonji suppresses viruses from multiplying. If the virus manages to sneak past the immune system and enters the body, studies have shown that Kalonji, and its active ingredients such as thymoquinone, are able to directly stop viruses, such as coronaviruses and others from multiplying. For example, a study published from University of Gaziantep, in Turkey demonstrated that administration of Kalonji extract to cells infected with coronavirus resulted in suppression of coronavirus multiplication and reduction of pathological protein production [105]. Antiviral activity of Kalonji was demonstrated in other studies, for example, viral hepatitis, and others [106].

Third. Kalonji protects the lungs from pathology. Kalonji was also reported by scholars to possess potent anti-inflammatory effects where its active ingredient thymoquinone suppressed effectively the lipopolysaccharide-induced inflammatory reactions and reduced significantly the concentration of nitric oxide, a marker of inflammation [107]. Moreover, Kalonji has been proven to suppress the pathological processes through blocking the activities of IL-1, IL-6, nuclear factor-κB [108], IL-1β, cyclooxygenase-1, prostaglandin-E2, prostaglandin-D2 [109], cyclocoxygenase-2, and TNF-α [110] that act as potent inflammatory mediators and were reported to play a major role in the pathogenesis of Coronavirus infection.

Fourth. Kalonji protects against sepsis/too much inflammation. In peer reviewed study from King Saud University, Riyadh, Saudi Arabia, scientists examined two sets of mice (n=12 per group), with parallel control groups, were acutely treated with thymoquinone (ingredient from Kalonji) intraperitoneal injections of 1.0 and 2.0 mg/kg body weight, and were subsequently challenged with endotoxin Gram-negative bacteria (LPS O111:B4). In another set of experiments, thymoquinone was administered at doses of 0.75 and 1.0 mg/kg/day for three consecutive days prior to sepsis induction with live Escherichia coli. Survival of various groups was computed, and renal, hepatic and sepsis markers were quantified. Thymoquinone reduced mortality by 80-90% and improved both renal and hepatic biomarker profiles. The concentrations of IL-1 α with 0.75 mg/kg thymoquinone dose was 310.8±70.93 and 428.3±71.32 pg/ml in the 1 mg/kg group as opposed to controls (1187.0±278.64 pg/ ml; P<0.05). Likewise, IL-10 levels decreased significantly with 0.75 mg/kg thymoquinone treatment compared to controls (2885.0±553.98 vs. 5505.2±333.96 pg/ml; P<0.01). Mice treated with thymoquinone also exhibited relatively lower levels of TNF-α and IL-2 (P values=0.1817 and 0.0851, respectively). This study gives strength to the potential clinical relevance of thymoquinone in sepsis-related morbidity and mortality reduction and suggests that human studies should be performed [111].

Sulforaphane [1-isothiocyanato-4-(methylsulfinyl)-butane], an isothiocyanate, is a chemopreventive photochemical which is a potent inducer of phase II enzyme involved in the detoxification of xenobiotics [112]. Sulforaphane is produced from the hydrolysis of glucoraphanin, the most abundant glucosinolate found in broccoli, and also present in other Brassicaceae [113]. Numerous studies have reported prevention of cancer [114-118], as well as cancer inhibitory properties of sulforaphane [119-124]. Importantly, this led to studies which demonstrated anti-inflammatory effects of this compound.

One of the fundamental features of inflammation is production of TNF-alpha from monocytic lineage cells. Numerous studies have shown that sulforaphane is capable of suppressing this fundamental initiator of inflammation, in part through blocking NF-kappa B translocation. For example, Lin et al. compared the anti-inflammatory effect of sulforaphane on LPS-stimulated inflammation in primary peritoneal macrophages derived from Nrf2 (+/+) and Nrf2 (−/−) mice. Pretreatment with sulforaphane in Nrf2 (+/+) primary peritoneal macrophages potently inhibited LPS-stimulated mRNA expression, protein expression and production of TNF-alpha, IL-1beta, COX-2 and iNOS. HO-1 expression was significantly augmented in LPS-stimulated Nrf2 (+/+) primary peritoneal macrophages by sulforaphane. Interestingly, the anti-inflammatory effect was attenuated in Nrf2 (−/−) primary peritoneal macrophages. We concluded that SFN exerts its anti-inflammatory activity mainly via activation of Nrf2 in mouse peritoneal macrophages [125]. In a similar study, LPS-challenged macrophages were observed for cytokine production with or without sulforaphane pretreatment. Macrophages were pre-incubated for 6 h with a wide range of concentrations of SFN (0 to 50 μM), and then treated with LPS for 24 h. Nitric oxide (NO) concentration and gene expression of different inflammatory mediators, i.e., interleukin (IL)-6, tumor necrosis factor (TNF)-α, and IL-1β, were measured. sulforaphane neither directly reacted with cytokines, nor with NO. To understand the mechanisms, the authors performed analyses of the expression of regulatory enzyme inducible nitic oxide synthase (iNOS), the transcription factor NF-E2-related factor 2 (Nrf2), and its enzyme heme-oxygenase (HO)-1. The results revealed that LPS increased significantly the expression of inflammatory cytokines and concentration of NO in non-treated cells. sulforaphane was able to prevent the expression of NO and cytokines through regulating inflammatory enzyme iNOS and activation of Nrf2/HO-1 signal transduction pathway [126]. These data are significant because studies have shown both TNF-alpha but also interleukin-6 are involved in pathology of COVID-19 [127-137]. The utilization of sulforaphane as a substitute for anti-IL-6 antibodies would be more economical and potentially without associated toxicity. Other studies have also demonstrated ability of sulforaphane to suppress IL-6 [138-140]. Interestingly, a clinical study was performed in 40 healthy overweight subjects (ClinicalTrials.gov ID NCT 03390855). Treatment phase consisted on the consumption of broccoli sprouts (30 g/day) during 10 weeks and the follow-up phase of 10 weeks of normal diet without consumption of these broccoli sprouts. Anthropometric parameters as body fat mass, body weight, and BMI were determined. Inflammation status was assessed by measuring levels of TNF-α, IL-6, IL-1β and C-reactive protein. IL-6 levels significantly decreased (mean values from 4.76 pg/mL to 2.11 pg/mL with 70 days of broccoli consumption, p<0.001) and during control phase the inflammatory levels were maintained at low grade (mean values from 1.20 pg/mL to 2.66 pg/mL, p<0.001). C-reactive protein significantly decreased as well [141].

An additional potential benefit of sulforaphane is its ability to protect lungs against damage. It is known that the major cause of lethality associated with COVID-19 is acute respiratory distress syndrome (ARDS). It was demonstrated that sulforaphane is effective in the endotoxin model of this condition. In one experiment, BALB/c mice were treated with sulforaphane (50 mg/kg) and 3 days later, ARDS was induced by the administration of LPS (5 mg/kg). The results revealed that sulforaphane significantly decreased lactate dehydrogenase (LDH) activity (as shown by LDH assay), the wet-to-dry ratio of the lungs and the serum levels of interleukin-6 (IL-6) and tumor necrosis factor-α (TNF-α) (measured by ELISA), as well as nuclear factor-κB protein expression in mice with LPS-induced ARDS. Moreover, treatment with sulforaphane significantly inhibited prostaglandin E2 (PGE2) production, and cyclooxygenase-2 (COX-2), matrix metalloproteinase-9 (MMP-9) protein expression (as shown by western blot analysis), as well as inducible nitric oxide synthase (iNOS) activity in mice with LPS-induced ALI. Lastly, the researchers reported pre-treatment with sulforaphane activated the nuclear factor-E2-related factor 2 (Nrf2)/antioxidant response element (ARE) pathway in the mice with LPS-induced ARDS [142].

EGCG is similar to sulforaphane in that it has been reported to possess cancer preventative properties. This compound has been shown to be one of the top therapeutic ingredients in green tea. It is known from epidemiologic studies that green tea consumption associates with chemoprotective effects against cancer [143-153]. In addition, similarly to sulforaphane, EGCG has been shown to inhibit inflammatory mediators. The first suggestion of this were studies shown suppression of the pro-inflammatory transcription factor NF-kappa B. In a detailed molecular study, EGCG, a potent antitumor agent with anti-inflammatory and antioxidant properties was shown to inhibit nitric oxide (NO) generation as a marker of activated macrophages. Inhibition of NO production was observed when cells were cotreated with EGCG and LPS. iNOS activity in soluble extracts of lipopolysaccharide-activated macrophages treated with EGCG (5 and 10 microM) for 6-24 hr was significantly lower than that in macrophages without EGCG treatment. Western blot, reverse transcription-polymerase chain reaction, and Northern blot analyses demonstrated that significantly reduced 130-kDa protein and 4.5-kb mRNA levels of iNOS were expressed in lipopolysaccharide-activated macrophages with EGCG compared with those without EGCG. Electrophoretic mobility shift assay indicated that EGCG blocked the activation of nuclear factor-kappaB, a transcription factor necessary for iNOS induction. EGCG also blocked disappearance of inhibitor kappaB from cytosolic fraction. These results suggest that EGCG decreases the activity and protein levels of iNOS by reducing the expression of iNOS mRNA and the reduction could occur through prevention of the binding of nuclear factor-kappaB to the iNOS promoter [154]. Another study supporting ability of EGCG to suppress NF-kappa B examined a model of atherosclerosis in which exposure of macrophage foam cells to TNF-α results in a downregulation of ABCA1 and a decrease in cholesterol efflux to apoA1, which is attenuated by pretreatment with EGCG. Moreover, rather than activating the Liver X receptor (LXR) pathway, inhibition of the TNF-α-induced nuclear factor-κB (NF-κB) activity is detected with EGCG treatment in cells. In order to inhibit the NF-κB activity, EGCG can promote the dissociation of the nuclear factor E2-related factor 2 (Nrf2)-Kelch-like ECH-associated protein 1 (Keap1) complex; when the released Nrf2 translocates to the nucleus and activates the transcription of genes containing an ARE element inhibition of NF-κB occurs and Keap1 is separated from the complex to directly interact with IKKβ and thus represses NF-κB function [155].

The anti-inflammatory effects of EGCG can be seen in the ability of this compound to potently inhibit IL-6, the COVID-19 associated cytokine, in a variety of inflammatory settings. For example, in a cardiac infarct model, rats were subjected to myocardial ischemia (30 min) and reperfusion (up to 2 h). Rats were treated with EGCG (10 mg/kg intravenously) or with vehicle at the end of the ischemia period followed by a continuous infusion (EGCG 10 mg/kg/h) during the reperfusion period. In vehicle-treated rats, extensive myocardial injury was associated with tissue neutrophil infiltration as evaluated by myeloperoxidase activity, and elevated levels of plasma creatine phosphokinase. Vehicle-treated rats also demonstrated increased plasma levels of interleukin-6. These events were associated with cytosol degradation of inhibitor kappaB-alpha, activation of IkappaB kinase, phosphorylation of c-Jun, and subsequent activation of nuclear factor-kappaB and activator protein-1 in the infarcted heart. In vivo treatment with EGCG reduced myocardial damage and myeloperoxidase activity. Plasma IL-6 and creatine phosphokinase levels were decreased after EGCG administration. This beneficial effect of EGCG was associated with reduction of nuclear factor-kB and activator protein-1 DNA binding [156]. In an inflammatory model of ulcerative colitis (UC) mice were randomly divided into four groups: Normal control, model (MD), 50 mg/kg/day EGCG treatment and 100 mg/kg/day EGCG treatment. The daily disease activity index (DAI) of the mice was recorded, changes in the organizational structure of the colon were observed and the spleen index (SI) was measured. In addition, levels of interleukin (IL)-6, IL-10, IL-17 and transforming growth factor (TGF)-β1 in the plasma and hypoxia-inducible factor (HIF)-1α and signal transducer and activator of transcription (STAT) 3 protein expression in colon tissues were evaluated. Compared with the MD group, the mice in the two EGCG treatment groups exhibited decreased DAIs and SIs and an attenuation in the colonic tissue erosion. EGCG could reduce the release of IL-6 and IL-17 and regulate the mouse splenic regulatory T-cell (Treg)/T helper 17 cell (Th17) ratio, while increasing the plasma levels of IL-10 and TGF-β1 and decreasing the HIF-1α and STATS protein expression in the colon. The experiments confirmed that EGCG treated mice with experimental colitis by inhibiting the release of IL-6 and regulating the body Treg/Th17 balance [157].

In patients with COVID-19, the ARDS associated with fatality resembles septic shock in many aspects, including DIC, fever, vascular leakage, and systemic inflammation. Wheeler et al. induced polymicrobial sepsis in male Sprague-Dawley rats (hemodynamic study) and C57BL6 mice (mortality study) via cecal ligation and double puncture (CL2P). Rodents were treated with either EGCG (10 mg/kg intraperitoneally) or vehicle at 1 and 6 h after CL2P and every 12 h thereafter. In the hemodynamic study, mean arterial blood pressure was monitored for 18 h, and rats were killed at 3, 6, and 18 h after CL2P. In the mortality study, survival was monitored for 72 h after CL2P in mice. In vehicle-treated rodents, CL2P was associated with profound hypotension and greater than 80% mortality rate. Epigallocatechin-3-gallate treatment significantly improved both the hypotension and survival [158].

A subsequent study by Li et al. showed intraperitoneal administration of EGCG protected mice against lethal endotoxemia, and rescued mice from lethal sepsis even when the first dose was given 24 hours after cecal ligation and puncture. The therapeutic effects were partly attributable to: 1) attenuation of systemic accumulation of proinflammatory mediator (e.g., HMGB1) and surrogate marker (e.g., IL-6 and KC) of lethal sepsis; and 2) suppression of HMGB 1-mediated inflammatory responses by preventing clustering of exogenous HMGB 1 on macrophage cell surface [159].

Finally, in a lung study, mice were treated with EGCG (10 mg/kg) intraperitoneally (ip) 1 h before LPS injection (10 mg/kg, ip). The results showed that EGCG attenuated LPS-induced ARDS as it decreased the changes in blood gases and reduced the histological lesions, wet-to-dry weight ratios, and myeloperoxidase (MPO) activity. In addition, EGCG significantly decreased the expression of pro-inflammatory cytokines tumor necrosis factor (TNF)-α, interleukin (IL)-1β, and IL-6 in the lung, serum, and bronchoalveolar lavage fluid, and alleviated the expression of TLR-4, MyD88, TRIF, and p-p65 in the lung tissue. In addition, it increased the expression of IκB-α and had no influence on the expression of p65. Collectively, these results demonstrated the protective effects of EGCG against LPS-induced ARDS in mice through its anti-inflammatory effect that may be attributed to the suppression of the activation of TLR 4-dependent NF-κB signaling pathways [160].

We disclose that the invention further provides nutraceutical compositions comprising the chemical compound of the invention, or a pharmaceutically acceptable salt or derivative thereof, together with one or more nutraceutically acceptable carriers, and, optionally, other therapeutic and/or prophylactic ingredients, known and used in the art. The carrier(s) must be “acceptable” in the sense of being compatible with the other ingredients of the formulation and not harmful to the recipient thereof. An oral composition can generally include an inert diluent or an edible carrier. The nutraceutical composition can comprise a functional food component or a nutrient component. The term “functional food” refers to a food which contains one or a combination of components which affects functions in the body so as to have positive cellular or physiological effects. The term “nutrient” refers to any substance that furnishes nourishment to an animal.

Pharmaceutical compositions of the invention may be those suitable for oral, rectal, bronchial, nasal, pulmonal, topical (including buccal and sub-lingual), transdermal, vaginal or parenteral (including cutaneous, subcutaneous, intramuscular, intraperitoneal, intravenous, intraarterial, intracerebral, intraocular injection or infusion) administration, or those in a form suitable for

administration by inhalation or insufflation, including powders and liquid aerosol administration, or by sustained release systems. Suitable examples of sustained release systems include semipermeable matrices of solid hydrophobic polymers containing the compound of the invention, which matrices may be in form of shaped articles, e.g. films or microcapsules.

The chemical compound of the invention, together with a conventional adjuvant, carrier, or diluent, may thus be placed into the form of pharmaceutical compositions and unit dosages thereof. Such forms include solids, and in particular tablets, filled capsules, powder and pellet forms, and liquids, in particular, aqueous or non-aqueous solutions, suspensions, emulsions, elixirs, and capsules filled with the same, all for oral use, suppositories for rectal administration, and sterile injectable solutions for parenteral use. Such pharmaceutical compositions and unit dosage forms thereof may comprise conventional ingredients in conventional proportions, with or without additional active compounds or principles, and such unit dosage forms may contain any suitable effective amount of the active ingredient commensurate with the intended daily dosage range to be employed.

The chemical compound of the present invention can be administered in a wide variety of oral and parenteral dosage forms. It will be obvious to those skilled in the art that the following dosage forms may comprise, as the active component, either a chemical compound of the invention or a pharmaceutically acceptable salt of a chemical compound of the invention.

For preparing pharmaceutical compositions from a chemical compound of the present invention, pharmaceutically acceptable carriers can be either solid or liquid. Solid form preparations include powders, tablets, pills, capsules, cachets, suppositories, and dispersible granules. A solid carrier can be one or more substances which may also act as diluents, flavoring agents, solubilizers, lubricants, suspending agents, binders, preservatives, tablet disintegrating agents, or an encapsulating material.

In powders, the carrier is a finely divided solid, which is in a mixture with the finely divided active component. In tablets, the active component is mixed with the carrier having the necessary binding capacity in suitable proportions and compacted in the shape and size desired.

The powders and tablets preferably contain from five or ten to about seventy percent of the active compound. Suitable carriers are magnesium carbonate, magnesium stearate, talc, sugar, lactose, pectin, dextrin, starch, gelatin, tragacanth, methylcellulose, sodium carboxymethylcellulose, a low melting wax, cocoa butter, and the like. The term “preparation” is intended to include the formulation of the active compound with encapsulating material as carrier providing a capsule in which the active component, with or without carriers, is surrounded by a carrier, which is thus in association with it. Similarly, cachets and lozenges are included. Tablets, powders, capsules, pills, cachets, and lozenges can be used as solid forms suitable for oral administration.

Liquid preparations include solutions, suspensions, and emulsions, for example, water or water-propylene glycol solutions. For example, parenteral injection liquid preparations can be formulated as solutions in aqueous polyethylene glycol solution. The chemical compound according to the present invention may thus be formulated for parenteral administration (e.g. by injection, for example bolus injection or continuous infusion) and may be presented in unit dose form in ampoules, pre-filled syringes, small volume infusion or in multi-dose containers with an added preservative. The compositions may take such forms as suspensions, solutions, or emulsions in oily or aqueous vehicles, and may contain formulation agents such as suspending, stabilizing and/or dispersing agents. Alternatively, the active ingredient may be in powder form, obtained by aseptic isolation of sterile solid or by lyophilization from solution, for constitution with a suitable vehicle, e.g. sterile, pyrogen-free water, before use.

Aqueous solutions suitable for oral use can be prepared by dissolving the active component in water and adding suitable colorants, flavors, stabilizing and thickening agents, as desired. Aqueous suspensions suitable for oral use can be made by dispersing the finely divided active component in water with viscous material, such as natural or synthetic gums, resins, methylcellulose, sodium carboxymethylcellulose, or other well-known suspending agents.

For topical administration to the epidermis the chemical compound of the invention may be formulated as ointments, creams, or lotions, or as a transdermal patch. Ointments and creams may, for example, be formulated with an aqueous or oily base with the addition of suitable thickening and/or gelling agents. Lotions may be formulated with an aqueous or oily base and will in general also contain one or more emulsifying agents, stabilizing agents, dispersing agents, suspending agents, thickening agents, or coloring agents.

Compositions suitable for topical administration in the mouth include lozenges comprising the active agent in a flavored base, usually sucrose and acacia or tragacanth; pastilles comprising the active ingredient in an inert base such as gelatin and glycerin or sucrose and acacia; and mouthwashes comprising the active ingredient in a suitable liquid carrier.

Solutions or suspensions are applied directly to the nasal cavity by conventional means, for example with a dropper, pipette, or spray. The compositions may be provided in single or multi-dose form. In compositions intended for administration to the respiratory tract, including intranasal compositions, the compound will generally have a small particle size for example of the order of 5 microns or less. Such a particle size may be obtained by means known in the art, for example by micronization.

The pharmaceutical preparations are preferably in unit dosage forms. In such form, the preparation is subdivided into unit doses containing appropriate quantities of the active component. The unit dosage form can be a packaged preparation, the package containing discrete quantities of preparation, such as packaged tablets, capsules, and powders in vials or ampoules. Also, the unit dosage form can be a capsule, tablet, cachet, or lozenge itself, or it can be the appropriate number of any of these in packaged form.

Tablets, capsules and lozenges for oral administration and liquids for intravenous administration and continuous infusion are preferred compositions. Solutions or suspensions for application to the nasal cavity or to the respiratory tract are preferred compositions. Transdermal patches for topical administration to the epidermis are preferred.

Details on techniques for formulation and administration may be found in the latest edition of Remington's Pharmaceutical Sciences (Maack Publishing Co., Easton, Pa.).

A therapeutically effective dose refers to that amount of active ingredient, which ameliorates the symptoms or condition. Therapeutic efficacy and toxicity, e.g. ED.sub.50 and LD.sub.50, may be determined by standard pharmacological procedures in cell cultures or experimental animals. The dose ratio between therapeutic and toxic effects is the therapeutic index and may be expressed by the ratio LD.sub.50/ED.sub.50. Pharmaceutical compositions exhibiting large therapeutic indexes are preferred.

The dosage of compound used in accordance with the invention varies depending on the compound and the condition being treated. The age, lean body weight, total weight, body surface area, and clinical condition of the recipient patient; and the experience and judgment of the clinician or practitioner administering the therapy are among the factors affecting the selected dosage. Other factors include the route of administration, the patient's medical history, the severity of the disease process, and the potency of the particular compound. The dose should be sufficient to ameliorate symptoms or signs of the disease treated without producing unacceptable toxicity to the patient. The dosage may be varied by titration of the dosage to the particular circumstances of this invention to produce the desired therapeutic effect. In one embodiment of the invention, co-administration of QuadraMune and ivermectin is utilized to augment NK cell number and activity.

### EXAMPLE

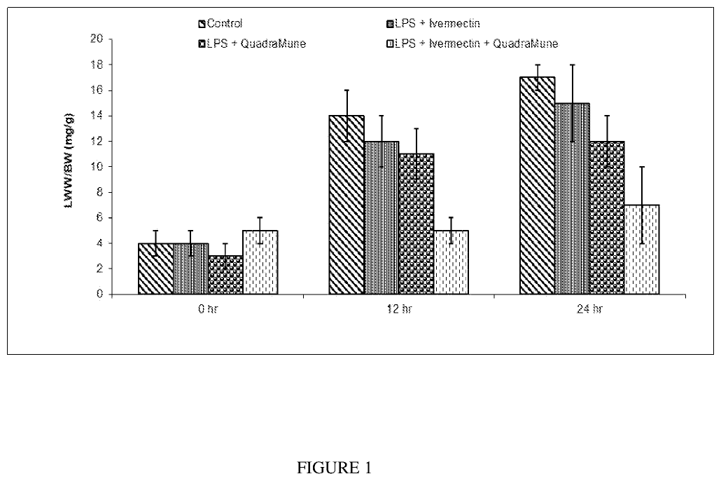

BABL/c mice, 5-7 weeks of age, females, were intraperitoneally injected with 50 mg/kg pentobarbital. Lipopolysaccharides (LPS) (5 mg/kg) (Sigma-Aldrich) was delivered to the lungs through a tracheostomy. Group 1 received LPS alone, Group 2, ivermectin orally at 2 mg/kg for at time of challenge, Group 3 received QuadraMune, and Group 4 received the combination. Lung wait was compared to whole body weight. Results are shown in FIG. 1.

### REFERENCES

1. Guzman-Martinez, O., et al., Potential Protection of Pre-Existent Antibodies to Human Coronavirus 229E on COVID-19 Severity. Int J Environ Res Public Health, 2021. 18(17).
2. Kaczmarczyk, L. S., et al., Corona and polio viruses are sensitive to short pulses of W-band gyrotron radiation. Environ Chem Lett, 2021: p. 1-6.
3. Imai, K., et al., Cross-reactive humoral immune responses against seasonal human coronaviruses in COVID-19 patients with different disease severities. Int J Infect Dis, 2021. 111: p. 68-75.
4. Mesel-Lemoine, M., et al., A human coronavirus responsible for the common cold massively kills dendritic cells but not monocytes. J Virol, 2012. 86(14): p. 7577-87.
5. van den Worm, S. H., et al., Reverse genetics of SARS-related coronavirus using vaccinia virus-based recombination. PLoS One, 2012. 7(3): p. e32857.
6. Friedman, N., et al., Transcriptomic profiling and genomic mutational analysis of Human coronavirus (HCoV)-229E-infected human cells. PLoS One, 2021. 16(2): p. e0247128.
7. Lynch, S. A., et al., Prevalence of Neutralising Antibodies to HCoV-NL63 in Healthy Adults in Australia. Viruses, 2021. 13(8).
8. Hukic, M., et al., Potential effect of previous HCoV-NL63 infection on the rate of infection and the clinical course of COVID-19. J Infect Dis, 2021.
9. Guo, W., et al., Glycan Nanostructures of Human Coronaviruses. Int J Nanomedicine, 2021. 16: p. 4813-4830.
10. Wondrak, G. T., et al., Solar simulated ultraviolet radiation inactivates HCoV-NL63 and SARS-CoV-2 coronaviruses at environmentally relevant doses. bioRxiv, 2021.
11. Ravindran, R., et al., Immune response dynamics in COVID-19 patients to SARS-CoV-2 and other human coronaviruses. PLoS One, 2021. 16(7): p. e0254367.
12. Sharwani, K., et al., Detection of serum cross-reactive antibodies and memory response to SARS-CoV-2 in pre pandemic and post-COVID-19 convalescent samples. J Infect Dis, 2021.
13. Li, Z., et al., Complicated pulmonary human coronavirus-NL63 infection after a second allogeneic hematopoietic stem cell transplantation for acute B-lymphocytic leukemia: A case report. Medicine (Baltimore), 2021. 100(25): p. e26446.
14. El-Senousy, W. M. and M. Shouman, Human Coronavirus NL 63 Among Other Respiratory Viruses in Clinical Specimens of Egyptian Children and Raw Sewage Samples. Food Environ Virol, 2021. 13(3): p. 322-328.
15. Lopez Alvarez, J. M., et al., Multiple-organ failure as a result of non-COVID-19 coronavirus infection. Arch Argent Pediatr, 2021. 119(3): p. e252-e255.
16. Otsuka, Y., et al., A patient with human coronavirus NL63 falsely diagnosed with COVID-19; Lesson learned for the importance of definitive diagnosis. J Infect Chemother, 2021. 27(7): p. 1126-1128.
17. Lineburg, K. E., et al., CD8(+) T cells specific for an immunodominant SARS-CoV-2 nucleocapsid epitope cross-react with selective seasonal coronaviruses. Immunity, 2021. 54(5): p. 1055-1065 e5.
18. Woldemeskel, B. A., C. C. Garliss, and J. N. Blankson, SARS-CoV-2 mRNA vaccines induce broad CD4+T cell responses that recognize SARS-CoV-2 variants and HCoV-NL63. J Clin Invest, 2021. 131(10).
19. Brown, E. L. and H. T. Essigmann, Original Antigenic Sin: the Downside of Immunological Memory and Implications for COVID-19. mSphere, 2021. 6(2).
20. Aldridge, R. W., et al., Seasonality and immunity to laboratory-confirmed seasonal coronaviruses (HCoV-NL63, HCoV-OC43, and HCoV-229E): results from the Flu Watch cohort study. Wellcome Open Res, 2020. 5: p. 52.
21. Saletti, G., et al., Older adults lack SARS CoV-2 cross-reactive T lymphocytes directed to human coronaviruses OC43 and NL63. Sci Rep, 2020. 10(1): p. 21447.
22. Guo, L., et al., Cross-reactive antibody against human coronavirus OC43 spike protein correlates with disease severity in COVID-19 patients: a retrospective study. Emerg Microbes Infect, 2021. 10(1): p. 664-676.
23. Georgakopoulou, V. E., et al., First Detection of Human Coronavirus HKU1 in Greece, in an Immunocompromised Patient With Severe Lower Respiratory Tract Infection. Acta Med Litu, 2021. 28(1): p. 121-126.
24. Burgess, H. M., et al., Targeting the m(6)A RNA modification pathway blocks SARS-CoV-2 and HCoV-OC43 replication. Genes Dev, 2021. 35(13-14): p. 1005-1019.
25. Esper, F., et al., Coronavirus HKU1 infection in the United States. Emerg Infect Dis, 2006. 12(5): p. 775-9.
26. Lau, S. K., et al., Coronavirus HKU1 and other coronavirus infections in Hong Kong. J Clin Microbiol, 2006. 44(6): p. 2063-71.
27. Gerna, G., et al., Human respiratory coronavirus HKU1 versus other coronavirus infections in Italian hospitalised patients. J Clin Virol, 2007. 38(3): p. 244-50.
28. Kupfer, B., et al., Two cases of severe obstructive pneumonia associated with an HKU1-like coronavirus. Eur J Med Res, 2007. 12(3): p. 134-8.
29. Zhao, Q., et al., Structure of the main protease from a global infectious human coronavirus, HCoV-HKU1. J Virol, 2008. 82(17): p. 8647-55.
30. Minosse, C., et al., Phylogenetic analysis of human coronavirus NL63 circulating in Italy. J Clin Virol, 2008. 43(1): p. 114-9.
31. Huang, P., et al., Nucleic acid visualization assay for Middle East Respiratory Syndrome Coronavirus (MERS-CoV) by targeting the UpE and N gene. PLoS Negl Trop Dis, 2021. 15(3): p. e0009227.
32. Rabets, A., et al., The Potential of Developing Pan-Coronaviral Antibodies to Spike Peptides in Convalescent COVID-19 Patients. Arch Immunol Ther Exp (Warsz), 2021. 69(1): p. 5.
33. Alshehri, M. A., et al., On the Prevalence and Potential Functionality of an Intrinsic Disorder in the MERS-CoV Proteome. Viruses, 2021. 13(2).
34. Park, B. K., et al., MERS-CoV and SARS-CoV-2 replication can be inhibited by targeting the interaction between the viral spike protein and the nucleocapsid protein. Theranostics, 2021. 11(8): p. 3853-3867.
35. Chen, J., et al., Development of A MERS-CoV Replicon Cell Line for Antiviral Screening. Virol Sin, 2021. 36(4): p. 730-735.
36. Abdelghany, T. M., et al., SARS-CoV-2, the other face to SARS-CoV and MERS-CoV: Future predictions. Biomed J, 2021. 44(1): p. 86-93.
37. Ansariniya, H., et al., Comparison of Immune Response between SARS, MERS, and COVID-19 Infection, Perspective on Vaccine Design and Development. Biomed Res Int, 2021. 2021: p. 8870425.
38. Saha, J., et al., A comparative genomics-based study of positive strand RNA viruses emphasizing on SARS-CoV-2 utilizing dinucleotide signature, codon usage and codon context analyses. Gene Rep, 2021. 23: p. 101055.
39. Lee, G. C., et al., Immunologic resilience and COVID-19 survival advantage. J Allergy Clin Immunol, 2021.
40. Marimuthu, A. K., et al., Utility of various inflammatory markers in predicting outcomes of hospitalized patients with COVID-19 pneumonia: A single-center experience. Lung India, 2021. 38(5): p. 448-453.
41. Herrera-Van Oostdam, A. S., et al., Immunometabolic signatures predict risk of progression to sepsis in COVID-19. PLoS One, 2021. 16(8): p. e0256784.
42. Saji, R., et al., Combining IL-6 and SARS-CoV-2 RNAaemia-based risk stratification for fatal outcomes of COVID-19. PLoS One, 2021. 16(8): p. e0256022.
43. Hotez, P. J., M. E. Bottazzi, and D. B. Corry, The potential role of Th17 immune responses in coronavirus immunopathology and vaccine-induced immune enhancement. Microbes Infect, 2020. 22(4-5): p. 165-167.
44. Casillo, G. M., et al., Could IL-17 represent a new therapeutic target for the treatment and/or management of COVID-19-related respiratory syndrome? Pharmacol Res, 2020. 156: p. 104791.
45. Pacha, O., M. A. Sallman, and S. E. Evans, COVID-19: a case for inhibiting IL-17? Nat Rev Immunol, 2020. 20(6): p. 345-346.
46. Raucci, F., et al., Interleukin-17A (IL-17A), a key molecule of innate and adaptive immunity, and its potential involvement in COVID-19-related thrombotic and vascular mechanisms. Autoimmun Rev, 2020. 19(7): p.
47. Ayhan, E., et al., Potential role of anti-interleukin-17 in COVID-19 treatment. Dermatol Ther, 2020. 33(4): p. e13715.
48. De Biasi, S., et al., Marked T cell activation, senescence, exhaustion and skewing towards TH17 in patients with COVID-19 pneumonia. Nat Commun, 2020. 11(1): p. 3434.
49. Orlov, M., et al., A Case for Targeting Th17 Cells and IL-17A in SARS-CoV-2 Infections. J Immunol, 2020. 205(4): p. 892-898.
50. Vatsalya, V., et al., Therapeutic Prospects for Th-17 Cell Immune Storm Syndrome and Neurological Symptoms in COVID-19: Thiamine Efficacy and Safety, In-vitro Evidence and Pharmacokinetic Profile. medRxiv, 2020.
51. Tu, W. J., et al., Clinicolaboratory study of 25 fatal cases of COVID-19 in Wuhan. Intensive Care Med, 2020. 46(6): p. 1117-1120.
52. Li, X., et al., Risk factors for severity and mortality in adult COVID-19 inpatients in Wuhan. J Allergy Clin Immunol, 2020. 146(1): p. 110-118.
53. Liu, P. P., et al., The Science Underlying COVID-19: Implications for the Cardiovascular System. Circulation, 2020. 142(1): p. 68-78.
54. Niedzwiedzka-Rystwej, P., et al., Interplay between Neutrophils, NETs and T-Cells in SARS-CoV-2 Infection—A Missing Piece of the Puzzle in the COVID-19 Pathogenesis? Cells, 2021. 10(7).
55. Banerjee, S. and A. K. Mahapatra, Systematic Review on Treatment Trials of Tocilizumab—A Repurposing Drug Against COVID-19. Rev Recent Clin Trials, 2021.
56. Dravid, A., et al., Combination therapy of Tocilizumab and steroid for management of COVID-19 associated cytokine release syndrome: A single center experience from Pune, Western India. Medicine (Baltimore), 2021. 100(29): p. e26705.
57. Sanchez-Rovira, P., et al., Early use of tocilizumab in patients with severe pneumonia secondary to severe acute respiratory syndrome coronavirus 2 infection and poor prognostic criteria: Impact on mortality rate and intensive care unit admission. Medicine (Baltimore), 2021. 100(29): p. e26533.
58. Phan, F., et al., Cardiac adipose tissue volume and IL-6 level at admission are complementary predictors of severity and short-term mortality in COVID-19 diabetic patients. Cardiovasc Diabetol, 2021. 20(1): p. 165.
59. Ferat-Osorio, E. and C. Lopez-Macias, Anti-interleukin 6 receptor antibody treatment in patients with COVID-19, is it key to reduce mortality? Rev Med Inst Mex Seguro Soc, 2020. 58(5): p. 541-542.
60. Zhang, C., et al., Cytokine release syndrome in severe COVID-19: interleukin-6 receptor antagonist tocilizumab may be the key to reduce mortality. Int J Antimicrob Agents, 2020. 55(5): p. 105954.
61. Luo, P., et al., Tocilizumab treatment in COVID-19: A single center experience. J Med Virol, 2020. 92(7): p. 814-818.
62. Dinarello, C. A., Proinflammatory cytokines. Chest, 2000. 118(2): p. 503-8.
63. Reka, G., et al., Impact of level of vitamin D in the body on the severity of COVID-19-review of the literature. Przegl Epidemiol, 2020. 74(4): p. 583-595.
64. Abdelhafiz, A. S., et al., Upregulation of FOXP3 is associated with severity of hypoxia and poor outcomes in COVID-19 patients. Virology, 2021. 563: p. 74-81.
65. Ferrer, P., et al., Association between pterostilbene and quercetin inhibits metastatic activity of B16 melanoma. Neoplasia, 2005. 7(1): p. 37-47.
66. Ferrer, P., et al., Nitric oxide mediates natural polyphenol-induced Bcl-2 down-regulation and activation of cell death in metastatic B16 melanoma. J Biol Chem, 2007. 282(5): p. 2880-90.
67. Zhang, L., et al., Pterostilbene protects vascular endothelial cells against oxidized low-density lipoprotein-induced apoptosis in vitro and in vivo. Apoptosis, 2012. 17(1): p. 25-36.
68. Park, S. H., et al., Pterostilbene, an Active Constituent of Blueberries, Stimulates Nitric Oxide Production via Activation of Endothelial Nitric Oxide Synthase in Human Umbilical Vein Endothelial Cells. Plant Foods Hum Nutr, 2015. 70(3): p. 263-8.
69. Chen, Z. W., et al., Pterostilbene protects against uraemia serum-induced endothelial cell damage via activation of Keap1/Nrf2/HO-1 signaling. Int Urol Nephrol, 2018. 50(3): p. 559-570.
70. Zhang, L., et al., Pterostilbene and its nicotinate derivative ameliorated vascular endothelial senescence and elicited endothelium-dependent relaxations via activation of sirtuin 1. Can J Physiol Pharmacol, 2021. 99(9): p. 900-909.
71. Malhotra, D., et al., Decline in NRF2-regulated antioxidants in chronic obstructive pulmonary disease lungs due to loss of its positive regulator, DJ-1. Am J Respir Crit Care Med, 2008. 178(6): p. 592-604.
72. Cho, H. Y., et al., Antiviral activity of Nrf2 in a murine model of respiratory syncytial virus disease. Am J Respir Crit Care Med, 2009. 179(2): p. 138-50.
73. Malhotra, D., et al., Heightened endoplasmic reticulum stress in the lungs of patients with chronic obstructive pulmonary disease: the role of Nrf2-regulated proteasomal activity. Am J Respir Crit Care Med, 2009. 180(12): p. 1196-207.
74. Michaeloudes, C., et al., Transforming growth factor-beta and nuclear factor E2-related factor 2 regulate antioxidant responses in airway smooth muscle cells: role in asthma. Am J Respir Crit Care Med, 2011. 184(8): p. 894-903.
75. McCormack, D. and D. McFadden, A review of pterostilbene antioxidant activity and disease modification. Oxid Med Cell Longev, 2013. 2013: p. 575482.
76. Paul, B., et al., Occurrence of resveratrol and pterostilbene in age-old darakchasava, an ayurvedic medicine from India. J Ethnopharmacol, 1999. 68(1-3): p. 71-6.
77. Kapetanovic, I. M., et al., Pharmacokinetics, oral bioavailability, and metabolic profile of resveratrol and its dimethylether analog, pterostilbene, in rats. Cancer Chemother Pharmacol, 2011. 68(3): p. 593-601.
78. Perecko, T., et al., Molecular targets of the natural antioxidant pterostilbene: effect on protein kinase C, caspase-3 and apoptosis in human neutrophils in vitro. Neuro Endocrinol Lett, 2010. 31 Suppl 2: p. 84-90.
79. Stivala, L. A., et al., Specific structural determinants are responsible for the antioxidant activity and the cell cycle effects of resveratrol. J Biol Chem, 2001. 276(25): p. 22586-94.
80. Athar, M., et al., Resveratrol: a review of preclinical studies for human cancer prevention. Toxicol Appl Pharmacol, 2007. 224(3): p. 274-83.
81. Bishayee, A., Cancer prevention and treatment with resveratrol: from rodent studies to clinical trials. Cancer Prey Res (Phila), 2009. 2(5): p. 409-18.
82. Hsu, C. L., et al., The inhibitory effect of pterostilbene on inflammatory responses during the interaction of 3T3-L1 adipocytes and RAW 264.7 macrophages. J Agric Food Chem, 2013. 61(3): p. 602-10.
83. McCormack, D., D. McDonald, and D. McFadden, Pterostilbene ameliorates tumor necrosis factor alpha-induced pancreatitis in vitro. J Surg Res, 2012. 178(1): p. 28-32.
84. Erasalo, H., et al., Natural Stilbenoids Have Anti-Inflammatory Properties in Vivo and Down-Regulate the Production of Inflammatory Mediators NO, IL6, and MCPI Possibly in a PI3K/Akt-Dependent Manner. J Nat Prod, 2018. 81(5): p. 1131-1142.
85. Allijn, I. E., et al., Head-to-Head Comparison of Anti-Inflammatory Performance of Known Natural Products In Vitro. PLoS One, 2016. 11(5): p. e0155325.
86. Meng, X. L., et al., Effects of resveratrol and its derivatives on lipopolysaccharide-induced microglial activation and their structure-activity relationships. Chem Biol Interact, 2008. 174(1): p. 51-9.
87. Swamy, S. M. and B. K. Tan, Cytotoxic and immunopotentiating effects of ethanolic extract of Nigella sativa L. seeds. J Ethnopharmacol, 2000. 70(1): p. 1-7.
88. Salem, M. L., F. Q. Alenzi, and W. Y. Attia, Thymoquinone, the active ingredient of Nigella sativa seeds, enhances survival and activity of antigen-specific CD8-positive T cells in vitro. Br J Biomed Sci, 2011. 68(3): p. 131-7.
89. Majdalawieh, A. F., R. Hmaidan, and R. I. Carr, Nigella sativa modulates splenocyte proliferation, Th1/Th2 cytokine profile, macrophage function and NK anti-tumor activity. J Ethnopharmacol, 2010. 131(2): p. 268-75.
90. Salomi, M. J., et al., Anti-cancer activity of nigella sativa. Anc Sci Life, 1989. 8(3-4): p. 262-6.
91. Salomi, N. J., et al., Antitumour principles from Nigella sativa seeds. Cancer Lett, 1992. 63(1): p. 41-6.
92. Ait Mbarek, L., et al., Anti-tumor properties of blackseed (Nigella sativa L.) extracts. Braz J Med Biol Res, 2007. 40(6): p. 839-47.
93. Amara, A. A., M. H. El-Masry, and H. H. Bogdady, Plant crude extracts could be the solution: extracts showing in vivo antitumorigenic activity. Pak J Pharm Sci, 2008. 21(2): p. 159-71.
94. Banerjee, S., et al., Review on molecular and therapeutic potential of thymoquinone in cancer. Nutr Cancer, 2010. 62(7): p. 938-46.
95. Khan, M. A., et al., Anticancer activities of Nigella sativa (black cumin). Afr J Tradit Complement Altern Med, 2011. 8(5 Suppl): p. 226-32.
96. Woo, C. C., et al., Thymoquinone: potential cure for inflammatory disorders and cancer. Biochem Pharmacol, 2012. 83(4): p. 443-51.
97. Lei, X., et al., Thymoquinone inhibits growth and augments 5-fluorouracil-induced apoptosis in gastric cancer cells both in vitro and in vivo. Biochem Biophys Res Commun, 2012. 417(2): p. 864-8.
98. Linjawi, S. A., et al., Evaluation of the protective effect of Nigella sativa extract and its primary active component thymoquinone against DMBA-induced breast cancer in female rats. Arch Med Sci, 2015. 11(1): p. 220-9.
99. Majdalawieh, A. F. and M. W. Fayyad, Recent advances on the anti-cancer properties of Nigella sativa, a widely used food additive. J Ayurveda Integr Med, 2016. 7(3): p. 173-180.
100. Majdalawieh, A. F., M. W. Fayyad, and G. K. Nasrallah, Anti-cancer properties and mechanisms of action of thymoquinone, the major active ingredient of Nigella sativa. Crit Rev Food Sci Nutr, 2017. 57(18): p. 3911-3928.
101. Mostofa, A. G. M., et al., Thymoquinone as a Potential Adjuvant Therapy for Cancer Treatment. Evidence from Preclinical Studies. Front Pharmacol, 2017. 8: p. 295.
102. Asaduzzaman Khan, M., et al., Thymoquinone, as an anticancer molecule: from basic research to clinical investigation. Oncotarget, 2017. 8(31): p. 51907-51919.
103. Imran, M., et al., Thymoquinone: A novel strategy to combat cancer: A review. Biomed Pharmacother, 2018. 106: p. 390-402.
104. Zhang, Y., et al., Thymoquinone inhibits the metastasis of renal cell cancer cells by inducing autophagy via AMPK/mTOR signaling pathway. Cancer Sci, 2018. 109(12): p. 3865-3873.
105. Ulasli, M., et al., The effects of Nigella sativa (Ns), Anthemis hyalina (Ah) and Citrus sinensis (Cs) extracts on the replication of coronavirus and the expression of TRP genes family. Mol Biol Rep, 2014. 41(3): p. 1703-11.
106. Ahmad, A., et al., A review on therapeutic potential of Nigella sativa: A miracle herb. Asian Pac J Trop Biomed, 2013. 3(5): p. 337-52.
107. Alemi, M., et al., Anti-inflammatory effect of seeds and callus of Nigella sativa L. extracts on mix glial cells with regard to their thymoquinone content. AAPS PharmSciTech, 2013. 14(1): p. 160-7.
108. Shuid, A. N., et al., Nigella sativa: A Potential Antiosteoporotic Agent. Evid Based Complement Alternat Med, 2012. 2012: p. 696230.
109. El Mezayen, R., et al., Effect of thymoquinone on cyclooxygenase expression and prostaglandin production in a mouse model of allergic airway inflammation. Immunol Lett, 2006. 106(1): p. 72-81.
110. Chehl, N., et al., Anti-inflammatory effects of the Nigella sativa seed extract, thymoquinone, in pancreatic cancer cells. HPB (Oxford), 2009. 11(5): p. 373-81.
111. Alkharfy, K. M., et al., The protective effect of thymoquinone against sepsis syndrome morbidity and mortality in mice. Int Immunopharmacol, 2011. 11(2): p. 250-4.
112. Shen, G., et al., Chemoprevention of familial adenomatous polyposis by natural dietary compounds sulforaphane and dibenzoylmethane alone and in combination in ApcMin/+mouse. Cancer Res, 2007. 67(20): p. 9937-44.
113. Zambrano, V., R. Bustos, and A. Mahn, Insights about stabilization of sulforaphane through microencapsulation. Heliyon, 2019. 5(11): p. e02951.
114. Steinkellner, H., et al., Effects of cruciferous vegetables and their constituents on drug metabolizing enzymes involved in the bioactivation of DNA-reactive dietary carcinogens. Mutat Res, 2001. 480-481: p. 285-97.
115. Fahey, J. W., Y. Zhang, and P. Talalay, Broccoli sprouts: an exceptionally rich source of inducers of enzymes that protect against chemical carcinogens. Proc Natl Acad Sci U S A, 1997. 94(19): p. 10367-72.
116. Solowiej, E., et al., Chemoprevention of cancerogenesis—the role of sulforaphane. Acta Pol Pharm, 2003. 60(1): p. 97-100.
117. Gills, J. J., et al., Sulforaphane prevents mouse skin tumorigenesis during the stage of promotion. Cancer Lett, 2006. 236(1): p. 72-9.
118. Myzak, M. C., et al., Sulforaphane inhibits histone deacetylase in vivo and suppresses tumorigenesis in Apc-minus mice. FASEB J, 2006. 20(3): p. 506-8.
119. Singh, A. V., et al., Sulforaphane induces caspase-mediated apoptosis in cultured PC-3 human prostate cancer cells and retards growth of PC-3 xenografts in vivo. Carcinogenesis, 2004. 25(1): p. 83-90.
120. Wang, L., et al., Targeting cell cycle machinery as a molecular mechanism of sulforaphane in prostate cancer prevention. Int J Oncol, 2004. 24(1): p. 187-92.
121. Pham, N. A., et al., The dietary isothiocyanate sulforaphane targets pathways of apoptosis, cell cycle arrest, and oxidative stress in human pancreatic cancer cells and inhibits tumor growth in severe combined immunodeficient mice. Mol Cancer Ther, 2004. 3(10): p. 1239-48.
122. Thejass, P. and G. Kuttan, Antimetastatic activity of Sulforaphane. Life Sci, 2006. 78(26): p. 3043-50.
123. Fimognari, C. and P. Hrelia, Sulforaphane as a promising molecule for fighting cancer. Mutat Res, 2007. 635(2-3): p. 90-104.
124. Li, Y., et al., Sulforaphane, a dietary component of broccoli/broccoli sprouts, inhibits breast cancer stem cells. Clin Cancer Res, 2010. 16(9): p. 2580-90.
125. Lin, W., et al., Sulforaphane suppressed LPS-induced inflammation in mouse peritoneal macrophages through Nrf2 dependent pathway. Biochem Pharmacol, 2008. 76(8): p. 967-73.
126. Ruhee, R. T., S. Ma, and K. Suzuki, Sulforaphane Protects Cells against Lipopolysaccharide-Stimulated Inflammation in Murine Macrophages. Antioxidants (Basel), 2019. 8(12).
127. Xu, X., et al., Effective treatment of severe COVID-19 patients with tocilizumab. Proc Natl Acad Sci U S A, 2020.
128. Liu, F., et al., Prognostic value of interleukin-6, C-reactive protein, and procalcitonin in patients with COVID-19. J Clin Virol, 2020. 127: p. 104370.
129. Aziz, M., R. Fatima, and R. Assaly, Elevated Interleukin-6 and Severe COVID-19: A Meta-Analysis. J Med Virol, 2020.
130. Chen, X., et al., Detectable serum SARS-CoV-2 viral load (RNAaemia) is closely correlated with drastically elevated interleukin 6 (IL-6) level in critically ill COVID-19 patients. Clin Infect Dis, 2020.
131. Zhang, C., et al., The cytokine release syndrome (CRS) of severe COVID-19 and Interleukin-6 receptor(IL-6R) antagonist Tocilizumab may be the key to reduce the mortality. Int J Antimicrob Agents, 2020: p. 105954.
132. Zhang, X., et al., First case of COVID-19 in a patient with multiple myeloma successfully treated with tocilizumab. Blood Adv, 2020. 4(7): p. 1307-1310.
133. McGonagle, D., et al., The Role of Cytokines including Interleukin-6 in COVID-19 induced Pneumonia and Macrophage Activation Syndrome-Like Disease. Autoimmun Rev, 2020: p. 102537.
134. Luo, P., et al., Tocilizumab treatment in COVID-19: A single center experience. J Med Virol, 2020.
135. Ulhaq, Z. S. and G. V. Soraya, Interleukin-6 as a potential biomarker of COVID-19 progression. Med Mal Infect, 2020.
136. Fu, B., X. Xu, and H. Wei, Why tocilizumab could be an effective treatment for severe COVID-19? J Transl Med, 2020. 18(1): p. 164.
137. Liu, B., et al., Can we use interleukin-6 (IL-6) blockade for coronavirus disease 2019 (COVID-19)-induced cytokine release syndrome (CRS)? J Autoimmun, 2020: p. 102452.
138. Eren, E., et al., Sulforaphane Inhibits Lipopolysaccharide-Induced Inflammation, Cytotoxicity, Oxidative Stress, and miR-155 Expression and Switches to Mox Phenotype through Activating Extracellular Signal-Regulated Kinase ½-Nuclear Factor Erythroid 2-Related Factor 2/Antioxidant Response Element Pathway in Murine Microglial Cells. Front Immunol, 2018. 9: p. 36.
139. Ma, T., et al., Sulforaphane, a Natural Isothiocyanate Compound, Improves Cardiac Function and Remodeling by Inhibiting Oxidative Stress and Inflammation in a Rabbit Model of Chronic Heart Failure. Med Sci Monit, 2018. 24: p. 1473-1483.
140. Liu, H., et al., Biomarker Exploration in Human Peripheral Blood Mononuclear Cells for Monitoring Sulforaphane Treatment Responses in Autism Spectrum Disorder. Sci Rep, 2020. 10(1): p. 5822.
141. Lopez-Chillon, M. T., et al., Effects of long-term consumption of broccoli sprouts on inflammatory markers in overweight subjects. Clin Nutr, 2019. 38(2): p. 745-752.
142. Qi, T., et al., Sulforaphane exerts anti-inflammatory effects against lipopolysaccharide-induced acute lung injury in mice through the Nrf2/ARE pathway. Int J Mol Med, 2016. 37(1): p. 182-8.
143. Dashwood, R. H., et al., Cancer chemopreventive mechanisms of tea against heterocyclic amine mutagens from cooked meat. Proc Soc Exp Biol Med, 1999. 220(4): p. 239-43.
144. Brown, M. D., Green tea (Camellia sinensis) extract and its possible role in the prevention of cancer. Altern Med Rev, 1999. 4(5): p. 360-70.
145. Banerjee, S., et al., Black tea polyphenols restrict benzopyrene-induced mouse lung cancer progression through inhibition of Cox-2 and induction of caspase-3 expression. Asian Pac J Cancer Prey, 2006. 7(4): p. 661-6.
146. Shimizu, M., Y. Shirakami, and H. Moriwaki, Targeting receptor tyrosine kinases for chemoprevention by green tea catechin, EGCG. Int J Mol Sci, 2008. 9(6): p. 1034-49.
147. Johnson, J. J., H. H. Bailey, and H. Mukhtar, Green tea polyphenols for prostate cancer chemoprevention: a translational perspective. Phytomedicine, 2010. 17(1): p. 3-13.
148. Kim, J. W., A. R. Amin, and D. M. Shin, Chemoprevention of head and neck cancer with green tea polyphenols. Cancer Prey Res (Phila), 2010. 3(8): p. 900-9.
149. Henning, S. M., P. Wang, and D. Heber, Chemopreventive effects of tea in prostate cancer: green tea versus black tea. Mol Nutr Food Res, 2011. 55(6): p. 905-20.
150. Du, G. J., et al., Epigallocatechin Gallate (EGCG) is the most effective cancer chemopreventive polyphenol in green tea. Nutrients, 2012. 4(11): p. 1679-91.
151. Henning, S. M., et al., Phenolic acid concentrations in plasma and urine from men consuming green or black tea and potential chemopreventive properties for colon cancer. Mol Nutr Food Res, 2013. 57(3): p. 483-93.
152. Schramm, L., Going Green: The Role of the Green Tea Component EGCG in Chemoprevention. J Carcinog Mutagen, 2013. 4(142): p. 1000142.
153. Rahmani, A. H., et al., Implications of Green Tea and Its Constituents in the Prevention of Cancer via the Modulation of Cell Signalling Pathway. Biomed Res Int, 2015. 2015: p. 925640.
154. Lin, Y. L. and J. K. Lin, (-)-Epigallocatechin-3-gallate blocks the induction of nitric oxide synthase by down-regulating lipopolysaccharide-induced activity of transcription factor nuclear factor-kappaB. Mol Pharmacol, 1997. 52(3): p. 465-72.
155. Jiang, J., et al., Epigallocatechin-3-gallate prevents TNF-alpha-induced NF-kappaB activation thereby upregulating ABCA1 via the Nrf2/Keap1 pathway in macrophage foam cells. Int J Mol Med, 2012. 29(5): p. 946-56.
156. Aneja, R., et al., Epigallocatechin, a green tea polyphenol, attenuates myocardial ischemia reperfusion injury in rats. Mol Med, 2004. 10(1-6): p. 55-62.
157. Xu, Z., et al., Epigallocatechin-3-gallate-induced inhibition of interleukin-6 release and adjustment of the regulatory T/T helper 17 cell balance in the treatment of colitis in mice. Exp Ther Med, 2015. 10(6): p. 2231-2238.
158. Wheeler, D. S., et al., The green tea polyphenol epigallocatechin-3-gallate improves systemic hemodynamics and survival in rodent models of polymicrobial sepsis. Shock, 2007. 28(3): p. 353-9.
159. Li, W., et al., A major ingredient of green tea rescues mice from lethal sepsis partly by inhibiting HMGB1. PLoS One, 2007. 2(11): p. e1153.
160. Wang, J., S. M. Fan, and J. Zhang, Epigallocatechin-3-gallate ameliorates lipopolysaccharide-induced acute lung injury by suppression of TLR4/NF-kappaB signaling activation. Braz J Med Biol Res, 2019. 52(7): p. e8092.

1. A method of augmenting the prophylactic and/or therapeutic effects of ivermectin on COVID-19 comprising administering said ivermectin together with one or more natural ingredients selected from the group consisting of: a) Green Tea and/or extract thereof; b) Blueberry and/or extract thereof; c) Nigella sativa and/or extract thereof; and d) broccoli and/or extract thereof.

2. The method of claim 1, wherein said green tea extract is epigallocatechin-3-gallate or an analogue thereof.

3. The method of claim 1, wherein said blueberry extract is pterostilbene or an analogue thereof.

4. The method of claim 1, wherein said Nigella sativa extract is thymoquinone or an analogue thereof.

5. The method of claim 1, wherein said broccoli extract is sulforaphane or an analogue thereof.

6. The method of claim 1, wherein said therapeutic combination is administered at a dosage and frequency sufficient to inhibit lung protective effects of ivermectin.

7. The method of claim 6, wherein said lung protective effects are reduction of inflammatory cytokines in the lung.

8. The method of claim 7, wherein said inflammatory cytokines are selected from the group consisting of: a) interleukin-1; b) interleukin-2; c) interleukin-5; d) interleukin-6; e) interleukin-8; f) interleukin-9; g) interleukin-11; h) interleukin-12; i) interleukin-15; j) interleukin-16; k) interleukin-17;1) interleukin-18; m) interleukin-21; n) interleukin-22; o) interleukin-23; p) interleukin-25; q) interleukin-27; r) interleukin-33; s) TNF-alpha; t) TNF-beta; u) interferon alpha; v) interferon beta; w) interferon gamma; x) interferon tau; y) interferon omega.

9. The method of claim 7, wherein said lung protective effect is reduction of neutrophil infiltration into the lung.

10. The method of claim 7, wherein said lung protective effect is reduction of T cell infiltration into the lung.

11. A method of reducing inflammation associated hypercoagulation states comprising administration of a therapeutic combination comprising: a) Green Tea and/or extract thereof; b) Blueberry and/or extract thereof; c) Nigella sativa and/or extract thereof; d) broccoli and/or extract thereof and e) ivermectin.

12. The method of claim 11, wherein said green tea extract is epigallocatechin-3-gallate or an analogue thereof.

13. The method of claim 11, wherein said blueberry extract is pterostilbene or an analogue thereof.

14. The method of claim 11, wherein said Nigella sativa extract is thymoquinone or an analogue thereof.

15. The method of claim 11, wherein said broccoli extract is sulforaphane or an analogue thereof.

16. The method of claim 11, wherein said therapeutic combination is administered at a dosage and frequency sufficient to inhibit tissue factor expression.

17. The method of claim 16, wherein said tissue factor expression is on the endothelium.

18. The method of claim 16, wherein said tissue factor expression is on microglia.

19. The method of claim 16, wherein said tissue factor expression is on the monocytes.

20. The method of claim 16, wherein said tissue factor expression is on pulmonary endothelium.
